(19) United States
(12) Patent Application Publication
(10) Pub. No.: US 2011/0166119 A1
(43) Pub. Date: Jul. 7, 2011

(54) Synergistic Combinations of Aztreonam with the Carbapenems Meropenem and Ertapenem

(75) Inventor: Kalyanasundaram Kasiviswanathan, Chennai (IN)

(73) Assignee: Glade Organics Private Limited, Mumbai (IN)

(21) Appl. No.: 13/063,754

(22) PCT Filed: Dec. 1, 2009
(86) PCT No.: PCT/IN2009/000698

§ 371 Date: Mar. 12, 2011

(30) Foreign Application Priority Data
Dec. 1, 2008 (IN) 2510/MUM/2008

(51) Int. Cl. A61K 31/397; A61P 31/04

(52) U.S. Cl. 514/210.13; 514/210.09

(57) ABSTRACT

A pharmaceutical composition comprising carbapenem and Aztreonam each in the dosage range of about 0.25 g to 0.75 g and pharmaceutically acceptable excipients for the prevention and treatment of infections caused by Pseudomonas aeruginosa, Acinetobacter baumannii, Klebsiella pneumoniae or MRSA (Methicillin Resistant Staphylococcus aureus).

### FIELD OF INVENTION

The present invention relates to a pharmaceutical composition comprising a carbapenem and a monobactum

### BACKGROUND OF THE INVENTION

Beta-lactam antibacterials (Penicillins and cephalosporins) have been widely prescribed to treat serious infections for nearly 60 years. No sooner than they were developed beta-lactams fell victim to the menace of resistance beta-lactamase enzyme production which is the primary mode of resistance to beta-lactam antibiotics. These enzymes are produced by some gram positive bacteria and virtually all gram negative bacteria. Beta-lactamases are a group of enzymes capable of hydrolyzing the 4-membered beta-lactam ring of beta-lactam antibiotics.

Generally, extended spectrum cephalosporins, such as the third generation cephalosporins, were thought to be resistant to hydrolysis by beta-lactamases (especially those produced by enteric bacilli such as E. coli and Klebsiella). However, in the mid 1980's it became evident that a new type of beta-lactamase was being produced by Klebsiella spp and in some cases by E. coli that could hydrolyse the extended spectrum cephalosporins. These new beta-lactamases have been collectively termed the ‘extended spectrum beta-lactamases’ (ESBL's). These beta-lactamases are variants of the common TEM-1, TEM-2, and SHV-1 enzymes.

All of these beta-lactamase enzymes are commonly found in the Enterobacteriaceae family. Normally, TEM-1, TEM-2, and SHV-1 enzymes confer high level resistance to early penicillins and low level resistance to first generation cephalosporins. Widespread use of third generation cephalosporins is believed to be the major cause of the mutations in these enzymes that has led to the emergence of the ESBLs. These enzymes mediate resistance to cefotaxime, ceftazidime and other broad spectrum cephalosporins.

The increase in ESBL-mediated resistance amongst E. coli and Klebsiella isolates worldwide make this a major public health threat. In the United States in 1990 to 1993 a survey of the intensive care units (ICUs) of 400 hospitals recorded an increase from 3.6% to 14.4% in ESBL producing strains of Klebsiella spp. By 1994 the Center for Disease Control and Prevention National Nosocomial Infections Surveillance System (NNIS) reported that 8% of Klebsiella spp had ESBLs with producers predominantly from a few large centers. A 1995-96 study in Richmond, Va. reported 1.5% of isolates produced ESBLs. In Europe as of 1995, ESBLs occur in 20%-25% of Klebsiella spp from patients in ICUs, although they have been found in up to 30%-40% in France. ESBLs are quite commonly encountered in the Asia-Pacific region. Rates vary greatly worldwide and within geographic areas and are rapidly changing over time.

Over the last 15 years, numerous outbreaks of infection with organisms producing extended-spectrum beta-lactamases (ESBLs) have been observed worldwide. The advent of ESBL producers has represented a great threat to the use of many classes of antibiotics, particularly cephalosporins.

Many ESBL producing organisms also express AmpC beta-lactamases and may be co-transferred with plasmids mediating aminoglycoside resistance. In addition, there is an increasing association between ESBL production and fluoroquinolone resistance. Currently, carbapenems are regarded as the drugs of choice for the treatment of infections caused by ESBL-producing organisms.

Carbapenems were first introduced in 1980 and are now frequently used as the last resort in treating serious infections caused by multidrug-resistant strains of gram negative bacilli. These antibiotics are stable to β-lactamase including the extended spectrum β-lactamase (ESBLs) and AmpC produced by gram negative bacilli. The carbapenems are a class of betalactamase antibiotics that differ from the penicillins by the substitution of a carbon atom for a sulfur atom and by the addition of a double bond to the five-membered ring of the penicillin nucleus.

The Carbapenem antibiotics Imipenem and Meropenem have the broadest antibacterial spectra of all the beta-lactams now available.

Ertapenem is a novel Carbapenem reported to have activity similar to that of meropenem against gram-positive bacteria, members of the family Enterobacteriaceae, and fastidious gram-negative bacteria but to be less active against Pseudomonas aeruginosa and Acinetobacter spp. Like meropenem, but unlike imipenem, ertapenem has a 1-b-methyl substituent and so does not require protection with an inhibitor of human renal dihydropeptidase I. Ertapenem's most distinguishing feature among carbapenems is a serum half-life of 4 to 4.5 h, which should allow once-daily administration, as with ceftriaxone. By contrast, Imipenem and meropenem must be administered three or four times daily have the broadest antibacterial spectra of all the beta-lactams now available.

Ertapenem is marketed for use in severe community-acquired infections, where non-fermenters are unlikely, and for intra-abdominal infections, community-acquired pneumonia and acute pelvic infection. In the European Union, it is licensed for skin and soft tissue infections and for complicated urinary infections.

Many E. coli isolates with CTX-M b-lactamases are susceptible only to Carbapenems.

However, as goes with the stereo typed history of antibacterials, all over the world, the last resort, or the “Magic Bullet” antibacterials called Carbapenems are also realized to be notified for gaining resistance.

The carbapenem resistance appears to be due to metallo-β-lactamase. There is evidence of the transfer of the multiple antibiotic resistance to other species including Escheichia coli, Enterobactr spp and Klebsiella spp. Multi-drug resistant (including carbapenem) in gram-negative bacteria pose a serious problem due to the lack of therapeutic options and the potential transfer of antibiotic resistance to other virulent pathogens.

The metallo-beta-lactamases which is in specific responsible for the carbepenem resistance, within the clinical sector, has been dramatic; the genes encoding metallo-beta-lactamases are often procured by class 1 (sometimes class 3) integrons, which, in turn, are embedded in transposons, resulting in a highly transmissible genetic apparatus. Moreover, other gene cassettes within the integrons often confer resistance to aminoglycosides, precluding their use as an alternative treatment. Thus far, the metallo-beta-lactamases encoded on transferable genes include IMP, VIM, SPM, and GIM and have been reported from 28 countries, and by the fact that there is currently no clinical inhibitor, nor is there likely to be for the foreseeable future.

There is also concern that extensive first-line use of Ertapenem as monotherapy will select cross-resistance to imipenem and meropenem, which are the last good defenses against many nosocomial infections caused by multiresistant pathogens. Particular concern is expressed about selection of carbapenem resistance in Pseudomonas aeruginosa, a species with a well-known propensity to develop resistance to imipenem and reduced susceptibility to meropenem via loss of porin OprD. P. aeruginosa also can develop reduced susceptibility to meropenem, along with resistance to most β-lactams except imipenem, via up-regulation of MexAB-OprM-mediated efflux.

Hence there is a need to combine the carbapenem with another antibiotic which can provide resistance to strains over a wide range and particularly against gram negative bacteria. The advantage of theses antibiotic combinations does not lie only in the synergistic outcome on the treatment part, but also in the reduced dosage (which results in economic advantage) of the components, in turn resulting reduction in the side effect profile, ultimately improving the compliance of the patents.

WO2007065288 discloses a medicament comprising a combination of monobactum antibiotic with β-lactamase inhibitors which are active against aerobic gram negative bacteria and in the form a generic disclosure teaches the combination of a carbapenem (i.e. Ertapenem) with a monobactum antibiotic (i.e. aztreonam) though the said document does not disclose the dosage range to be used for the above combination.

The present invention aims to provide a composition comprising a combination of Crabapenem and Aztreonam which shows synergistic effect against a wide range of strains.

### OBJECT OF THE INVENTION

It is an object of the present invention to provide a pharmaceutical composition which shows synergistic effect against a wide range of strains.

It is another object of the present invention to provide a pharmaceutical composition that is a combination of Ertapenem with Aztreonam which shows synergistic effect against a wide range of strains and particularly against gram negative bacteria strains.

It is another object of the present invention to provide a pharmaceutical composition that is a combination of Meropenem with Aztreonam which shows synergistic effect against a wide range of strains and particularly against gram negative bacteria strains.

It is another object of the present invention to provide a pharmaceutical composition which is useful as an ultimate empirical therapy in the ICU based antibiotic therapy protocols.

### SUMMARY OF THE INVENTION

A pharmaceutical composition comprising carbapenem and Aztreonam each in the dosage range of about 0.25 g to 0.75 g and pharmaceutically acceptable excipients.

### DETAILED DESCRIPTION OF THE INVENTION

Antimicrobial combinations are employed not only to broaden the spectrum of coverage but also to get the benefit of preventing the emergence of resistant strains in world of antimicrobials. The present invention provides a pharmaceutical antimicrobial combination of two beta lactam antibiotics which binds to the complementary Penicillin-binding proteins.

Meropenem is an ultra-broad spectrum injectable antibiotic used to treat a wide variety of infections, including meningitis and pneumonia. It is a beta-lactam and belongs to the subgroup of carbapenem, similar to imipenem and ertapenem.

Ertapenem which is structurally similar to Meropenem is an atypical Carbapenem and is effective against Gram negative bacteria. Ertapenem also has clinically useful activity against anaerobic bacteria

Aztreonam is a synthetic monocyclic beta-lactam antibiotic (a monobactum) originally isolated from Chromobacterium violaceum. It is resistant to some beta-lactamases, but is inactivated by extended-spectrum beta-lactamases.

The synergistic outcome of the present innovation of the carbapenems like Meropenem as well as Ertapenem with monobactums such as Aztreonam could be effectively exploited as a first-line therapy.

The combinations are also active against a variety of bacterial organisms. They are active against aerobic Gram-negative bacteria that do not produce β-lactamases, including Klebsiella pneumoniae, Pseudomonas for example P. aeruginosa and Acienetobacter for example A. baumannii. The above combination shows synergistic activity against strains of the above organisms that do produce β-lactamases.

The dosage of the active compound can depend on a variety of factors such as mode of administration, age of the individual. Preferred dosages for the active ingredients of the pharmaceutical combination according to the present invention are therapeutically effective dosages. Normally in case of intravenous mode, an approximate dosage of 0.25 to 0.75 g of each drug is preferred.

The combinations of the present invention may be administered intravenously or in the form of a powder.

The excipients to be used for the present invention are selected from sodium bicarbonate, sodium hydroxide and L-arginine.

Surprisingly it is found that the combinations of the present invention not only disclose effectiveness as a first line therapy, but is also effective as a second line therapy and can be termed as an ‘ultimate empirical therapy’ in the ICU based antibiotic therapy protocols.

The person skilled in the art is fully enabled to select a relevant test model to prove the efficacy of a combination of the present invention in the hereinafter indicated indications. The advantages of the present combinations are demonstrated via MIC data (Minimum inhibitory concentration) on a variety of strains via Checkerboard assay technique. The isobologram so produced indicate the synergistic effect of the present combination.

The following examples illustrate the invention described above and are not intended to restrict the scope of this invention in any way.

### Examples

### Biological Evaluation

### Experimental Data on MIC's of Selective Antibacterial Agents Against Organisms—Notorious for Resistance Issues Around the Globe

The combination of carbapenem with Aztreonam (i.e. Ertapenem+Aztreonam and Meropenem+Aztreonam) was tested for a variety of proportionate drug concentrations, with the checker board technique and an isobologram was constructed. The synergistic outcome of the combination was recorded as a concave curve. The synergistic value of the combination was displayed by a concentration of only 50% of the MIC of each drug.

Antimicrobial activity of the compounds and of their combinations was determined against a selection of organisms as described below:

### Method for Testing Antimicrobial Activity of Drugs:

The checkerboard array was performed in order to find the MIC's of selected antibacterial agents against the strains of Pseudomonas aeruginosa, Acinetobacter baumannii, Klebsiella pneumoniae and MRSA (Methicillin Resistant Staphylococcus aureus).

### Minimum Inhibitory Concentration (Checkerboard Method)

1. A total of 50 μl of Mueller-Hinton broth was distributed into each well of the micro dilution plates.
2. The first antibiotic (A) of the combination was serially twofold diluted along the Y-axis, while the second antibiotic (B) was diluted along the X-axis.
3. An inoculum equal to a 0.5 McFarland turbidity standard was prepared from each culture in sterile saline.
4. Each micro titer well was inoculated with 100 μl of a bacterial inoculum and the plate was loaded on ELISA plate reader (37° C. for 24 hrs).
   Formula for ΣFIC (Fractional inhibitory concentration):

ΣFIC=FIC A+FIC B,

Where FIC A is the MIC of drug A in the combination/MIC of drug A alone,

FIC B is the MIC of drug B in the combination/MIC of drug B alone.

The combination is considered synergistic when the ΣFIC is ≦0.5, indifferent when the ΣFIC is >0.5 to <2, and antagonistic when the ΣFIC is ≧2.

### Observations:

The MIC values (in mcg/ml) of representative compounds and their combinations are listed in Table 1, Table 2, Table 3 and table 4 against strains of Pseudomonas aeruginosa, Klebsiella pneumoniae, Acinetobacter baumannii and Methicillin Resistant Staphylococcus aureus (MRSA).

TABLE 1
Culture: Pseudomonas aeruginosa (PA1 - ESBL producing)

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 40000 | − | − | − | − | − | − | − | − | 312.5 | − | − | − | − |
| (mcg/ml) | B | 20000 | − | − | − | − | − | − | − | − | 156.25 | − | − | − | − |
| | C | 10000 | − | − | − | − | − | − | − | − | 78.12 | − | − | − | − |
| | D | 5000 | − | − | − | − | − | − | − | − | 0 | + | − | − | − |
| | | | | | | | | | | | | 0 | 39.06 | 78.12 | 156.25 |
| | E | 2500 | − | − | − | − | − | − | − | − | 39.06 | − | − | − | − |
| | F | 1250 | − | − | − | − | − | − | − | − | 19.53 | + | − | − | − |
| | | | | | | | | | | | | | | (0.5, 1) | |
| | G | 625 | − | − | − | − | − | − | − | − | 9.76 | + | − | − | − |
| | | | | | | | | | | | | | | | (2, 0.249) |
| | H | 0 | + | − | − | − | − | − | − | − | 0 | + | + | − | − |
| | | | 0 | 312.5 | 25 | 1250 | 2500 | 5000 | 10000 | 20000 | | 0 | 4.88 | 9.76 | 19.53 |

Ertapenem (mcg/ml)

### Minimum Inhibitory Concentration

$$Aztreonam \text{:} 39.06 mcg \text{/} ml$$

$$Ertapenem \text{:} 9.76 mcg \text{/} ml$$

$$Aztreonam + Ertapenem \text{:} 9.76 mcg \text{/} ml + 4.88 mcg \text{/} ml$$

$$\begin{matrix}Σ FIC = FIC Aztreonam + FIC Ertapenem \\ = \frac{9.76}{39.06} + \frac{4.88}{9.76} \\ = 0.2498 + 0.5 = 0.7498\end{matrix}$$

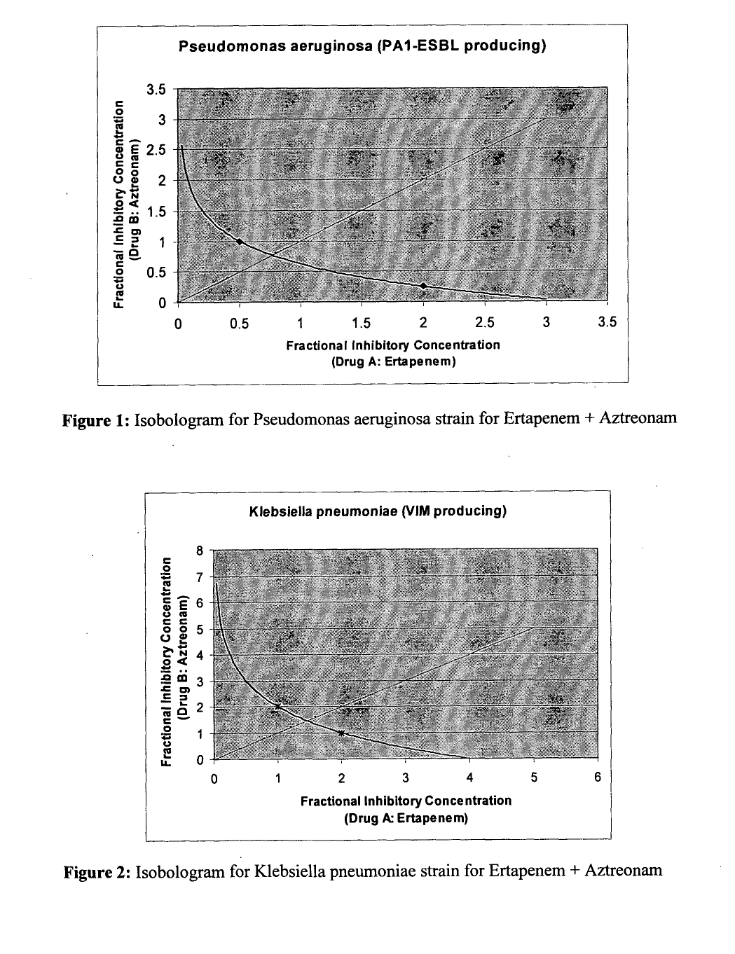

The above calculations imply that MIC for Aztreonam when taken individually corresponds to 39.06 which reduce to 9.76 in combination. Further MIC for Ertapenem when taken individually corresponds to 9.76 which reduces to 4.88 in combination. The FIC values as calculated above are used to draw the isobologram which proves the synergistic effect shown by the combination as compared to when taken alone. The isobologram as plotted with the below coefficients is as shown in FIG. 1.

### Effect of Combination of Two Antimicrobial Drugs

| | | FIC of Ertapenem | | |
|---|---|---|---|---|
| | | (x-co-ordinate) | | |
| | | 19.53 | 9.76 | 4.88 |
| FIC of | 39.06 | | | (0.5, 1.0) |
| Aztreonam | 19.53 | | (1.0, 0.5) | |
| (y-co- | 9.76 | (2.0, 0.249) | | |
| ordinate) | | | | |

TABLE 2
Culture: Klebsiella pneumoniae (VIM producing)

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 625 | + | − | − | − | − | − | − | − | 4.88 | + | + | − | − |
| (mcg/ml) | B | 312.5 | + | − | − | − | − | − | − | − | 2.44 | + | + | − | − |
| | | | | | | | | | | | | | | (1, 2) | |
| | C | 156.25 | + | − | − | − | − | − | − | − | 1.22 | + | + | − | − |
| | | | | | | | | | | | | | | | (2, 1) |
| | D | 78.12 | + | − | − | − | − | − | − | − | 0 | + | + | + | + |
| | | | | | | | | | | | | 0 | 0.61 | 1.22 | 2.44 |
| | E | 39.06 | + | − | − | − | − | − | − | − | 0.61 | + | + | + | + |
| | F | 19.53 | + | − | − | − | − | − | − | − | 0.305 | + | + | + | + |
| | G | 9.76 | + | − | − | − | − | − | − | − | 0.152 | + | + | + | + |
| | H | 0 | + | − | − | − | − | − | − | − | 0 | + | + | + | + |
| | | | 0 | 4.88 | 9.76 | 19.53 | 39.06 | 78.12 | 156.25 | 312.5 | | 0 | 0.076 | 0.152 | 0.305 |

Ertapenem (mcg/ml)

### Minimum Inhibitory Concentration

$$Aztreonam \text{:} 1250 mcg \text{/} ml$$

$$Ertapenem \text{:} 4.88 mcg \text{/} ml$$

$$Aztreonam + Ertapenem \text{:} 1.22 mcg \text{/} ml + 1.22 mcg \text{/} ml$$

$$\begin{matrix}Σ FIC = FIC Aztreonam + FIC Ertapenem \\ = \frac{1.22}{1250} + \frac{1.22}{4.88} \\ = 0.000976 + 0.25 = 0.250976\end{matrix}$$

The above calculations imply that MIC for Aztreonam when taken individually corresponds to 1250 which reduce to 1.22 in combination. Further MIC for Ertapenem when taken individually corresponds to 1.22 which reduce to 4.88 in combination. The FIC values as calculated above are used to draw the isobologram which proves the synergistic effect shown by the combination as compared to when taken alone. The isobologram as plotted with the below coefficients is as shown in FIG. 2.

### Effect of Combination of Two Antimicrobial Drugs

| | FIC of Ertapenem | | | | |
|---|---|---|---|---|---|
| | (x-co-ordinate) | | | | |
| | | 1.22 | 2.44 | 4.88 | 9.76 |
| FIC of | 2.44 | (0.25, 0.00195) | | | |
| Aztreonam | 4.88 | | (0.5, 0.0039) | | |
| (y-co- | 9.76 | | | (1.0, | |
| ordinate) | | | | 0.0078) | |
| | 19.53 | | | | (2.0, |
| | | | | | 0.016) |

TABLE 3
Culture: Acinetobacter baumanii

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 40000 | − | − | − | − | − | − | − | − | 312.5 | + | − | − | − |
| (mcg/ml) | B | 20000 | − | − | − | − | − | − | − | − | 156.25 | + | − | − | − |
| | | | | | | | | | | | | | | (0.25, 0.25) | |
| | C | 10000 | − | − | − | − | − | − | − | − | 78.12 | + | | − | − |
| | | | | | | | | | | | | | | | (0.5, 0.12) |
| | D | 5000 | + | − | − | − | − | − | − | − | 0 | + | + | + | + |
| | | | | | | | | | | | | 0 | 0.61 | 1.22 | 2.44 |
| | E | 2500 | + | − | − | − | − | − | − | − | 39.06 | + | + | + | + |
| | F | 1250 | + | − | − | − | − | − | − | − | 19.53 | + | + | + | + |
| | G | 625 | + | − | − | − | − | − | − | − | 9.76 | + | + | + | + |
| | H | 0 | + | + | − | − | − | − | − | − | 0 | + | + | + | + |
| | | | 0 | 4.88 | 9.76 | 19.53 | 39.06 | 78.12 | 156.25 | 312.5 | | 0 | 0.076 | 0.152 | 0.305 |

Ertapenem (mcg/ml)

### Minimum Inhibitory Concentration

$$Aztreonam \text{:} 10000 mcg \text{/} ml$$

$$Ertapenem \text{:} 9.76 mcg \text{/} ml$$

$$Aztreonam + Ertapenem \text{:} 78.12 mcg \text{/} ml + 0.61 mcg \text{/} ml$$

$$\begin{matrix}Σ FIC = FIC Aztreonam + FIC Ertapenem \\ = \frac{78.12}{10000} + \frac{0.61}{9.76} \\ = 0.07812 + 0.0625 = 0.14062\end{matrix}$$

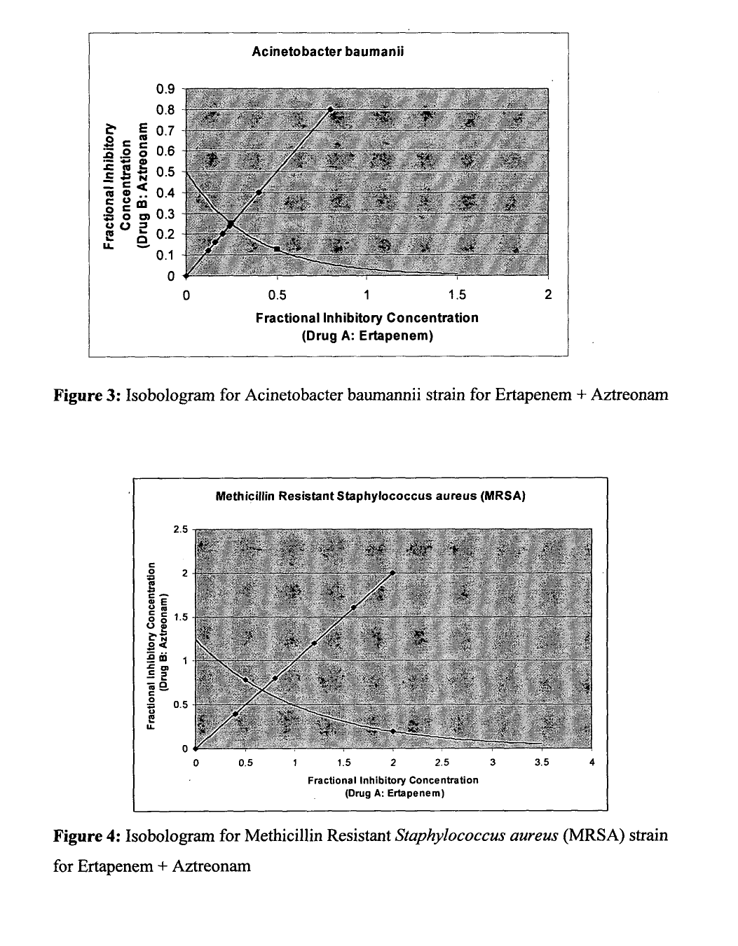

The above calculations imply that MIC for Aztreonam when taken individually corresponds to 10000 which reduce to 78.12 in combination. Further MIC for Ertapenem when taken individually corresponds to 9.76 which reduce to 0.61 in combination. The FIC values as calculated above are used to draw the isobologram which proves the synergistic effect shown by the combination as compared to when taken alone. The isobologram as plotted with the below coefficients is as shown in FIG. 3.

### Effect of Combination of Two Antimicrobial Drugs

| | FIC of Ertapenem | | | |
|---|---|---|---|---|
| | (x-co-ordinate) | | | |
| | | 0.61 | 1.22 | 2.44 |
| FIC of Aztreonam | 78.12 | | | (0.25, 0.0078) |
| (y-co-ordinate) | 156.25 | | (0.125, 0.0156) | |
| | 312.5 | (0.0625, | | |
| | | 0.0313) | | |

TABLE 4
Culture: Methicillin Resistant Staphylococcus aureus (MRSA)

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 40000 | − | − | − | − | − | − | − | − | 312.5 | + | − | − | − |
| (mcg/ml) | B | 20000 | − | − | − | − | − | − | − | − | 156.25 | + | − | − | − |
| | C | 10000 | − | − | − | − | − | − | − | − | 78.12 | + | − | − | − |
| | D | 5000 | − | − | − | − | − | − | − | − | 0 | + | − | − | − |
| | | | | | | | | | | | | 0 | 39.06 | 78.12 | 156.25 |
| | E | 2500 | + | − | − | − | − | − | − | − | 39.06 | + | − | − | − |
| | F | 1250 | + | − | − | − | − | − | − | − | 19.53 | + | − | − | − |
| | G | 625 | + | − | − | − | − | − | − | − | 9.76 | + | − | − | − |
| | H | 0 | + | − | − | − | − | − | − | − | 0 | + | + | − | − |
| | | | 0 | 312.5 | 625 | 2500 | 5000 | 10000 | 20000 | | | 0 | 4.88 | 9.76 | 19.53 |

Ertapenem (mcg/ml)

### Minimum Inhibitory Concentration

$$Aztreonam \text{:} 5000 mcg \text{/} ml$$

$$Ertapenem \text{:} 9.76 mcg \text{/} ml$$

$$Aztreonam + Ertapenem \text{:} 9.76 mcg \text{/} ml + 4.88 mcg \text{/} ml$$

$$\begin{matrix}Σ FIC = FIC Aztreonam + FIC Ertapenem \\ = \frac{9.76}{5000} + \frac{4.88}{9.76} \\ = 0.001952 + 0.5 = 0.501952\end{matrix}$$

The above calculations imply that MIC for Aztreonam when taken individually corresponds to 5000 which reduce to 9.76 in combination. Further MIC for Ertapenem when taken individually corresponds to 9.76 which reduce to 4.88 in combination. The FIC values as calculated above are used to draw the isobologram which proves the synergistic effect shown by the combination as compared to when taken alone. The isobologram as plotted with the below coefficients is as shown in FIG. 4.

### Effect of Combination of Two Antimicrobial Drugs

| | FIC of Ertapenem | | | |
|---|---|---|---|---|
| | (x-co-ordinate) | | | |
| | | 4.88 | 9.76 | 19.53 |
| FIC of | 9.76 | | | (2, 0.001952) |
| Aztreonam | 19.53 | | (1, 0.0039) | |
| (y-co-ordinate) | 39.06 | (0.5, 0.0078) | | |

From table 1, table 2, table 3 and table 4 and the isobologram, the synergistic effect of the combination in comparison to that as taken as individually can be observed. Thus the combination of Aztreonam with Ertapenem drastically reduces the MIC value which ultimately reduces the amount of actives used in the combination.

The combination of Meropenem with Aztreonam also shows the same synergistic effect as shown by Ertapenem with Aztreonam.

TABLE 5
Culture: Klebsiella pneumoniae (VIM producing)

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 20000 | − | − | − | − | − | − | − | − | 156.25 | + | − | − | − |
| (mcg/ml) | B | 10000 | − | − | − | − | − | − | − | − | 78.12 | + | − | − | − |
| | C | 5000 | − | − | − | − | − | − | − | − | 39.06 | + | − | − | − |
| | D | 2500 | − | − | − | − | − | − | − | − | 0 | + | + | − | − |
| | | | | | | | | | | | | 0 | 0.390 | 0.781 | 1.562 |
| | E | 1250 | − | − | − | − | − | − | − | − | 19.53 | + | + | − | − |
| | F | 625 | + | − | − | − | − | − | − | − | 9.76 | + | + | − | − |
| | G | 312.5 | + | − | − | − | − | − | − | − | 4.88 | + | + | + | + |
| | H | 0 | + | − | − | − | − | − | − | − | 0 | + | + | + | + |
| | | | 0 | 3.125 | 6.25 | 12.5 | 25 | 50 | 100 | 200 | | 0 | 0.0488 | 0.097 | 0.195 |

Meropenem(mcg/ml)

### Minimum Inhibitory Concentration

$$Aztreonam \text{:} 1250 mcg \text{/} ml$$

$$M eropenem \text{:} 0.781 mcg \text{/} ml$$

$$Aztreonam + Meropenem \text{:} 9.76 mcg \text{/} ml + 0.097 mcg \text{/} ml$$

$$\begin{matrix}Σ FIC = FIC Aztreonam + FIC Meropenem \\ = \frac{9.76}{1250} + \frac{0.097}{0.781} \\ = 0.0078 + 0.1242 = 0.132\end{matrix}$$

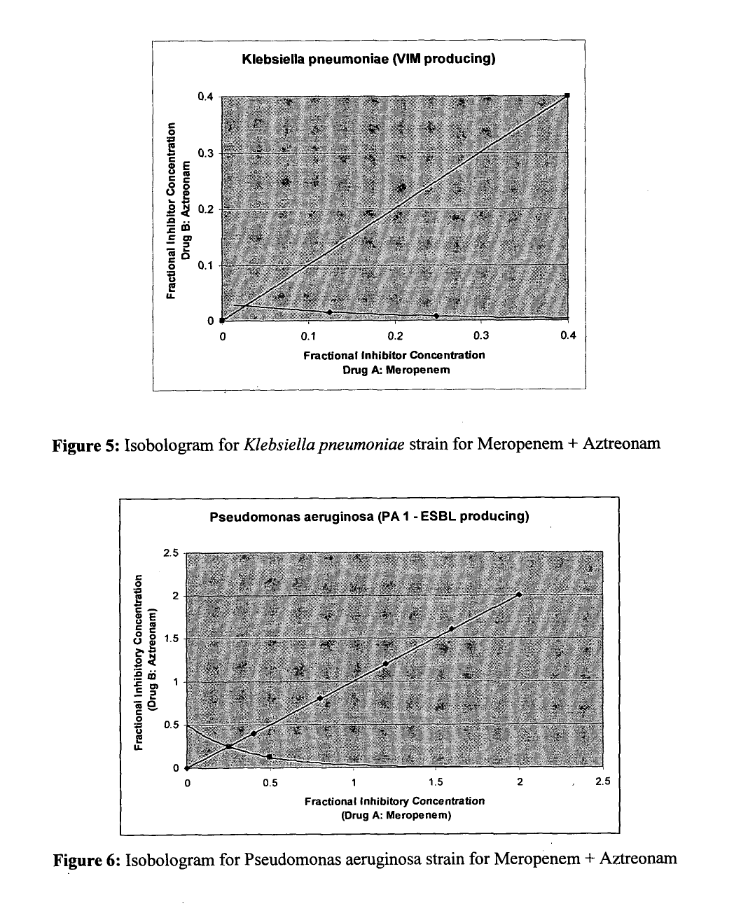

The above calculations imply that MIC for Aztreonam when taken individually corresponds to 1200 which reduce to 9.76 in combination. Further MIC for Meropenem when taken individually corresponds to 0.781 which reduce to 0.097 in combination. The FIC values as calculated above are used to draw the isobologram which proves the synergistic effect shown by the combination as compared to when taken alone. The isobologram as plotted with the below coefficients is as shown in FIG. 5.

### Effect of Combination of Two Antimicrobial Drugs

| | FIC of Meropenem | | |
|---|---|---|---|
| | (x-co-ordinate) | | |
| | | 0.195 | 0.097 |
| FIC of Aztreonam | 9.76 | (0.2496, 0.078) | |
| (y-coordinate) | 19.53 | | (0.124, 0.016) |

TABLE 6
Culture: Pseudomonas aeruginosa ATCC 27853

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 20000 | − | − | − | − | − | − | − | − | 156.25 | − | − | − | − |
| (mcg/ml) | B | 10000 | − | − | − | − | − | − | − | − | 78.12 | − | − | − | − |
| | C | 5000 | − | − | − | − | − | − | − | − | 39.06 | − | − | − | − |
| | D | 2500 | − | − | − | − | − | − | − | − | 0 | + | − | − | − |
| | | | | | | | | | | | | 0 | 0.390 | 0.781 | 1.562 |
| | E | 1250 | − | − | − | − | − | − | − | − | 19.53 | + | − | − | − |
| | F | 625 | − | − | − | − | − | − | − | − | 9.76 | + | − | − | − |
| | G | 312.5 | − | − | − | − | − | − | − | − | 4.88 | + | − | − | − |
| | H | 0 | + | − | − | − | − | − | − | − | 0 | + | + | + | − |
| | | | 0 | 3.125 | 6.25 | 12.5 | 25 | 50 | 100 | 200 | | 0 | 0.0488 | 0.097 | 0.195 |

Meropenem(mcg/ml)

### Minimum Inhibitory Concentration

$$Aztreonam \text{:} 39.06 mcg \text{/} ml$$

$$M eropenem \text{:} 0.195 mcg \text{/} ml$$

$$Aztreonam + Meropenem \text{:} 4.88 mcg \text{/} ml + 0.0488 mcg \text{/} ml$$

$$\begin{matrix}Σ FIC = FIC Aztreonam + FIC Meropenem \\ = \frac{4.88}{39.06} + \frac{0.0488}{0.195} \\ = 0.1249 + 0.2502 = 0.3751\end{matrix}$$

The above calculations imply that MIC for Aztreonam when taken individually corresponds to 39.06 which reduce to 4.88 in combination. Further MIC for Meropenem when taken individually corresponds to 0.195 which reduce to 0.0488 in combination. The FIC values as calculated above are used to draw the isobologram which proves the synergistic effect shown by the combination as compared to when taken alone. The isobologram as plotted with the below coefficients is as shown in FIG. 6.

### Effect of Combination of Two Antimicrobial Drugs

| | FIC of Meropenem | | | |
|---|---|---|---|---|
| | (x-co-ordinate) | | | |
| | | 0.195 | 0.097 | 0.0488 |
| FIC of Aztreonam | 4.88 | (1.0, 0.125) | | |
| (y-co-ordinate) | 9.76 | | (0.497, 0.25) | |
| | 19.53 | | | (0.25, 0.5) |

TABLE 7
Culture: Methicillin Resistant Staphylococcus aureus (MRSA)

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 20000 | − | − | − | − | − | − | − | − | 156.25 | + | − | − | − |
| (mcg/ml) | B | 10000 | − | − | − | − | − | − | − | − | 78.12 | + | − | − | − |
| | C | 5000 | − | − | − | − | − | − | − | − | 39.06 | + | − | − | − |
| | D | 2500 | + | − | − | − | − | − | − | − | 0 | + | − | − | − |
| | | | | | | | | | | | | 0 | 48.82 | 97.65 | 195.3 |
| | E | 1250 | + | − | − | − | − | − | − | − | 19.53 | + | − | − | − |
| | F | 625 | + | − | − | − | − | − | − | − | 9.76 | + | + | − | − |
| | G | 312.5 | + | − | − | − | − | − | − | − | 4.88 | + | + | − | − |
| | H | 0 | + | − | − | − | − | − | − | − | 0 | + | + | − | − |
| | | | 0 | 390.6 | 781.2 | 1562.5 | 3125 | 6250 | 12500 | 25000 | | 0 | 6.1 | 12.2 | 24.41 |

Meropenem (mcg/ml)

### Minimum Inhibitory Concentration

$$Aztreonam \text{:} 5000 mcg \text{/} ml$$

$$M eropenem \text{:} 12.2 mcg \text{/} ml$$

$$Aztreonam + Meropenem \text{:} 19.53 mcg \text{/} ml + 6.1 mcg \text{/} ml$$

$$\begin{matrix}Σ FIC = FIC Aztreonam + FIC Meropenem \\ = \frac{19.53}{5000} + \frac{6.1}{12.2} \\ = 0.003906 + 0.5 = 0.503906\end{matrix}$$

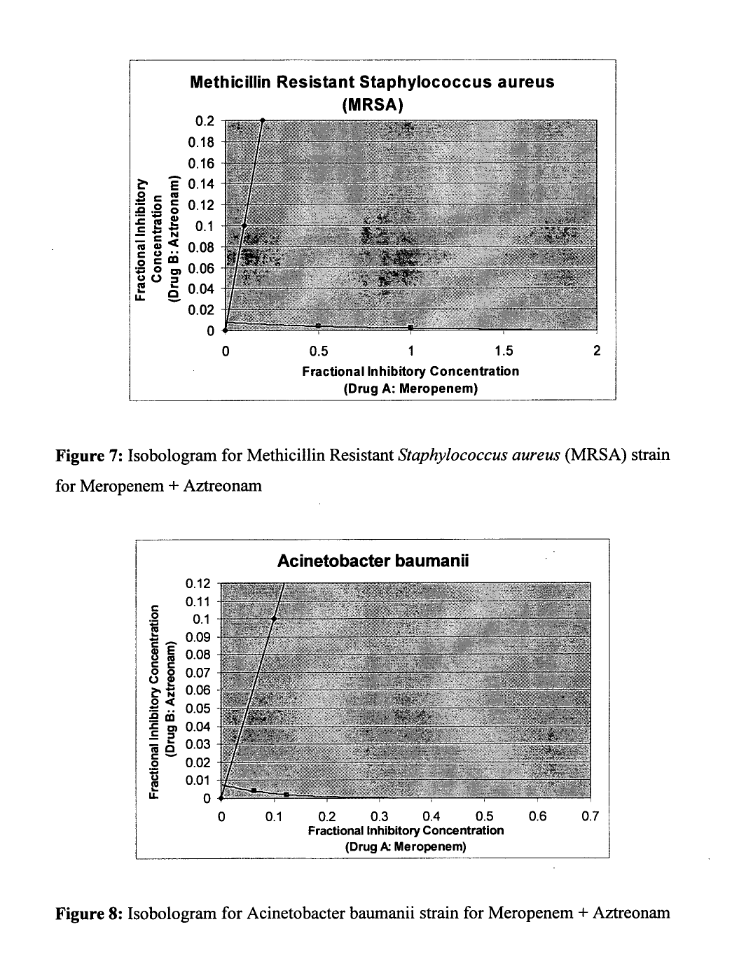

The above calculations imply that MIC for Aztreonam when taken individually corresponds to 5000 which reduce to 19.53 in combination. Further MIC for Meropenem when taken individually corresponds to 12.2 which reduce to 6.1 in combination. The FIC values as calculated above are used to draw the isobologram which proves the synergistic effect shown by the combination as compared to when taken alone. The isobologram as plotted with the below coefficients is as shown in FIG. 7.

### Effect of Combination of Two Antimicrobial Drugs

| | FIC of Meropenem | | |
|---|---|---|---|
| | (x-co-ordinate) | | |
| | | 12.2 | 6.1 |
| FIC of Aztreonam | 9.76 | (1, 0.001952) | |
| (y-co-ordinate) | 19.53 | | (0.5, 0.039) |

TABLE 8
Culture: Acinetobacter baumanii

| | | | 1 | 2 | 3 | 4 | 5 | 6 | 7 | 8 | | 9 | 10 | 11 | 12 |
|---|---|---|---|---|---|---|---|---|---|---|---|---|---|---|---|
| Aztreonam | A | 20000 | − | − | − | − | − | − | − | − | 156.25 | + | − | − | − |
| (mcg/ml) | B | 10000 | − | − | − | − | − | − | − | − | 78.12 | + | − | − | − |
| | C | 5000 | − | − | − | − | − | − | − | − | 39.06 | + | − | − | − |
| | D | 2500 | − | − | − | − | − | − | − | − | 0 | + | + | + | + |
| | | | | | | | | | | | | 0 | 4.88 | 9.76 | 19.53 |
| | E | 1250 | − | − | − | − | − | − | − | − | 19.53 | + | + | − | − |
| | F | 625 | − | − | − | − | − | − | − | − | 9.76 | + | + | − | − |
| | G | 312.5 | − | − | − | − | − | − | − | − | 4.88 | + | + | − | − |
| | H | 0 | + | − | − | − | − | − | − | − | 0 | + | + | + | + |
| | | | 0 | 39.06 | 78.12 | 156.25 | 312.5 | 625 | 1250 | 2500 | | 0 | 0.61 | 1.22 | 2.44 |

Meropenem(mcg/ml)

### Minimum Inhibitory Concentration

$$Aztreonam \text{:} 2500 mcg \text{/} ml$$

$$M eropenem \text{:} 19.53 mcg \text{/} ml$$

$$Aztreonam + Meropenem \text{:} 4.88 mcg \text{/} ml + 1.22 mcg \text{/} ml$$

$$\begin{matrix}Σ FIC = FIC Aztreonam + FIC Meropenem \\ = \frac{4.88}{5000} + \frac{1.22}{19.53} \\ = 0.000976 + 0.062 = 0.062976\end{matrix}$$

The above calculations imply that MIC for Aztreonam when taken individually corresponds to 5000 which reduce to 4.88 in combination. Further MIC for Meropenem when taken individually corresponds to 19.53 which reduce to 1.22 in combination. The FIC values as calculated above are used to draw the isobologram which proves the synergistic effect shown by the combination as compared to when taken alone. The isobologram as plotted with the below coefficients is as shown in FIG. 8.

### Effect of Combination of Two Antimicrobial Drugs

| | FIC of Meropenem | | |
|---|---|---|---|
| | (x-co-ordinate) | | |
| | | 1.22 | 2.44 |
| FIC of Aztreonam | 4.88 | | (0.125, 0.00195) |
| (y-co-ordinate) | 9.76 | (0.0625, 0.0039) | |

From table 5, table 6, table 7 and table 8 and the isobologram, the synergistic effect of the combination in comparison to that as taken as individually can be observed. Thus the combination of Aztreonam with Meropenem drastically reduces the MIC value which ultimately reduces the amount of actives used in the combination.

1. A pharmaceutical composition comprising carbapenem and Aztreonam each in a dosage range of about 0.25 g to 0.75 g and pharmaceutically acceptable excipients.

2. The pharmaceutical composition according to claim 1, wherein the carbapenem is selected from meropenem or ertapenem.

3. The pharmaceutical composition according to claim 1, wherein the pharmaceutically acceptable excipients is selected from sodium bicarbonate, sodium hydroxide, and L-arginine.

4. The pharmaceutical composition according to claim 1, for use in the prevention or treatment of infections caused by Pseudomonas aeruginosa, Acinetobacter baumannii, Klebsiella pneumoniae or MRSA (Methicillin Resistant Staphylococcus aureus).

5. A method of treatment or prevention of a disease condition caused by Pseudomonas aeruginosa, Acinetobacter baumannii, Klebsiella pneumoniae or MRSA (Methicillin Resistant Staphylococcus aureus) which comprises administering an effective amount of a pharmaceutical composition comprising carbapenem and Aztreonam and pharmaceutically acceptable excipients.

6. The method according to claim 5, wherein the carbapenem is present in a dosage range of about 0.25 g to 0.75 g.

7. The method according to claim 5, wherein Aztreonam is present in a dosage range of about 0.25 g to 0.75 g.

8. The method according to claim 5, wherein the carbapenem is selected from meropenem or ertapenem.

9. The method according to claim 5, wherein the pharmaceutically acceptable excipients is selected from sodium bicarbonate, sodium hydroxide, and L-arginine.

10. The pharmaceutical composition according to claim 2, for use in the prevention or treatment of infections caused by Pseudomonas aeruginosa, Acinetobacter baumannii, Klebsiella pneumoniae or MRSA (Methicillin Resistant Staphylococcus aureus).

11. The pharmaceutical composition according to claim 3, for use in the prevention or treatment of infections caused by Pseudomonas aeruginosa, Acinetobacter baumannii, Klebsiella pneumoniae or MRSA (Methicillin Resistant Staphylococcus aureus).
